(19) United States
(12) Patent Application Publication
(10) Pub. No.: US 2020/0196956 A1
(43) Pub. Date: Jun. 25, 2020

(54) METHODS AND SYSTEMS FOR INERTIAL MEASUREMENT OF PRESSURE MANAGEMENT MOVEMENTS

(71) Applicant: INVACARE CORPORATION, Elyria, OH (US)

(72) Inventors: Gary A.J. DELLOW, Christchurch (NZ); Agnetha KOREVAAR, Christchurch (NZ)

(21) Appl. No.: 16/643,012

(22) PCT Filed: Aug. 28, 2018
(86) PCT No.: PCT/US2018/048301

§ 371 Date: Feb. 28, 2020

Related U.S. Application Data
(60) Provisional application No. 62/551,016, filed on Aug. 28, 2017.

(51) Int. Cl. A61B 5/00; A61B 5/11; A61G 5/10

(52) U.S. Cl. CPC A61B 5/6894; A61B 5/1116; A61B 5/6801; A61B 5/1121; A61G 2205/60; A61G 5/10; A61B 5/74; A61G 2203/36; A61B 5/0002

(57) ABSTRACT

Exemplary systems and methods associated with pressure management system for a user of a wheelchair. The systems and methods utilize an apparatus attached directly to the user for detecting and transmitting the inertial movement of the user. Various methods of generating pressure relief data and information associated with pressure points/pressure point relief of the user based on the actual movements of the user are disclosed.

### RELATED APPLICATIONS

This application claims priority to, and the benefits of, U.S. provisional application Ser. No. 62/551,016 filed on Aug. 28, 2017, which is incorporated by reference herein in full.

### TECHNICAL FIELD

The present invention relates generally to methods and systems for pressure management of a wheelchair user. In particular, detecting and utilizing actual user movements to determine when to communicate alerts to the user for sufficient or insufficient pressure relief. This invention may also be used to provide short, medium and long term data to the wheelchair user and/or clinician for review against other users, recommended standards and/or personal goals. The invention also provides data that can be used for clinical research into the relationship between movement in a chair and a user's risk of developing a pressure ulcer and/or the relative effectiveness of various pressure relief motions.

### BACKGROUND OF THE INVENTION

It is well known that physically impaired individuals with such disabilities as general weakness, immobility, spinal cord injury, muscular dystrophy, multiple sclerosis, cerebral palsy, arthritis, etc. need the assistance of a wheelchair to be mobile. Wheelchairs, which may be of the type manufactured by Invacare Corporation of Elyria, Ohio, for example, generally include user support surfaces for supporting a user while in the wheelchair. For example, a seat mounted on the wheelchair forms a user support surface for the user to sit on. A seat back forms a user support surface for the user's back. A pair of arms and a pair of legs may be mounted on the wheelchair to form user support surfaces for the user's arms and legs, respectively.

Potential pressure points occur in areas where the user's body makes contact with the wheelchair support surfaces. These pressure points can result in pressure ulcers over time due to prolonged pressure without adequate pressure relief. User movement can alleviate the pressure at a pressure point and allow for at least partial tissue reperfusion.

### SUMMARY

According to one aspect of the present invention, an apparatus for detecting and transmitting inertial movement of a user of a wheelchair includes at least one movement sensor configured to be attached to the user while the user is supported by the wheelchair, where the movement sensor detects inertial movement of the user and generates movement data and a wireless interface device in operative communication with the at least one movement sensor, where the wireless interface device transmits the movement data wirelessly to a receiver associated with the apparatus.

The descriptions of the invention do not limit the words used in the claims in any way or the scope of the claims or invention. The words used in the claims have all of their full ordinary meanings.

### BRIEF DESCRIPTION OF THE DRAWINGS

In the accompanying drawings, which are incorporated in and constitute a part of the specification, embodiments of the invention are illustrated, which, together with a general description of the invention given above, and the detailed description given below, serve to exemplify embodiments of this invention.

FIG. 1A is a side view drawing of an exemplary manual wheelchair and a user.

FIG. 1B is a front view drawing of the exemplary manual wheelchair and the user.

FIG. 2 is a block diagram of an exemplary pressure management system associated with a wheelchair.

FIG. 3 is a block diagram of an exemplary pressure management system associated with a powered wheelchair.

FIG. 4 is a block diagram of an exemplary method for detecting and transmitting inertial movement.

FIG. 5 is a block diagram of an exemplary method for generating pressure management information.

FIG. 6 is a block diagram of an exemplary method for generating pressure relief data.

FIG. 7 is a block diagram of an exemplary method for generating and communicating pressure relief information.

### DESCRIPTION

The following includes definitions of exemplary terms used throughout the disclosure. Both singular and plural forms of all terms fall within each meaning:

“Circuit” or “circuitry,” as used herein includes, but is not limited to, hardware, firmware, software or combinations of each to perform a function(s) or an action(s). For example, based on a desired feature or need, a circuit may include a software controlled microprocessor, discrete logic such as an application specific integrated circuit (ASIC), or other programmed logic device. A circuit may also be fully embodied as software. As used herein, “circuit” is considered synonymous with “logic.”

“Controller,” as used herein includes, but is not limited to, any circuit or device that coordinates and controls the operation of one or more input or output devices. For example, a controller can include a device having one or more processors, microprocessors, or central processing units (CPUs) capable of being programmed to perform input or output functions.

“Logic,” as used herein includes, but is not limited to, hardware, firmware, software or combinations of each to perform a function(s) or an action(s), or to cause a function or action from another component. For example, based on a desired application or need, logic may include a software controlled microprocessor, discrete logic such as an application specific integrated circuit (ASIC), or other programmed logic device. Logic may also be fully embodied as software. As used herein, “logic” is considered synonymous with “circuit.”

“Operative communication” or “circuit communication,” as used herein includes, but is not limited to, a communicative relationship between devices, logic, or circuits, including mechanical and pneumatic relationships. Direct electrical, electromagnetic, and optical connections and indirect electrical, electromagnetic, and optical connections are examples of such communications. Linkages, gears, chains, push rods, cams, keys, attaching hardware, and other components facilitating mechanical connections are also examples of such communications. Pneumatic devices and interconnecting pneumatic tubing may also contribute to operative communications. Two devices are in operative communication if an action from one causes an effect in the other, regardless of whether the action is modified by some other device. For example, two devices separated by one or more of the following: i) amplifiers, ii) filters, iii) transformers, iv) optical isolators, v) digital or analog buffers, vi) analog integrators, vii) other electronic circuitry, viii) fiber optic transceivers, ix) Bluetooth communications links, x) 802.11 and 802.15 communications links, xi) satellite communication links, xii) near-field communication, and xiii) other wireless communication links. As another example, an electromagnetic sensor is in operative communication with a signal if it receives electromagnetic radiation from the signal. As a final example, two devices not directly connected to each other, but both capable of interfacing with a third device, e.g., a central processing unit (CPU), are in operative communication.

“Processor,” as used herein includes, but is not limited to, one or more of virtually any number of processor systems or stand-alone processors, such as microprocessors, microcontrollers, central processing units (CPUs), and digital signal processors (DSPs), in any combination. The processor may be associated with various other circuits that support operation of the processor, such as random access memory (RAM), read-only memory (ROM), programmable read-only memory (PROM), erasable programmable read-only memory (EPROM), clocks, decoders, memory controllers, or interrupt controllers, etc. These support circuits may be internal or external to the processor or its associated electronic packaging. The support circuits are in operative communication with the processor. The support circuits are not necessarily shown separate from the processor in block diagrams or other drawings.

“Signal,” as used herein includes, but is not limited to, one or more electrical signals, including analog or digital signals, one or more computer instructions, a bit or bit stream, or the like.

“Software,” as used herein includes, but is not limited to, one or more computer readable or executable instructions that cause a computer or other electronic device to perform functions, actions, or behave in a desired manner. The instructions may be embodied in various forms such as applications (apps), routines, algorithms, modules or programs including separate applications or code from dynamically linked libraries. Software may also be implemented in various forms such as a stand-alone program, a function call, a servlet, an applet, instructions stored in a memory, part of an operating system, or other types of executable instructions. It will be appreciated by one of ordinary skill in the art that the form of software is dependent on, for example, requirements of a desired application, the environment it runs on, or the desires of a designer/programmer or the like.

While the above exemplary definitions have been provided, it is Applicant's intention that the broadest reasonable interpretation consistent with this specification be used for these and other terms.

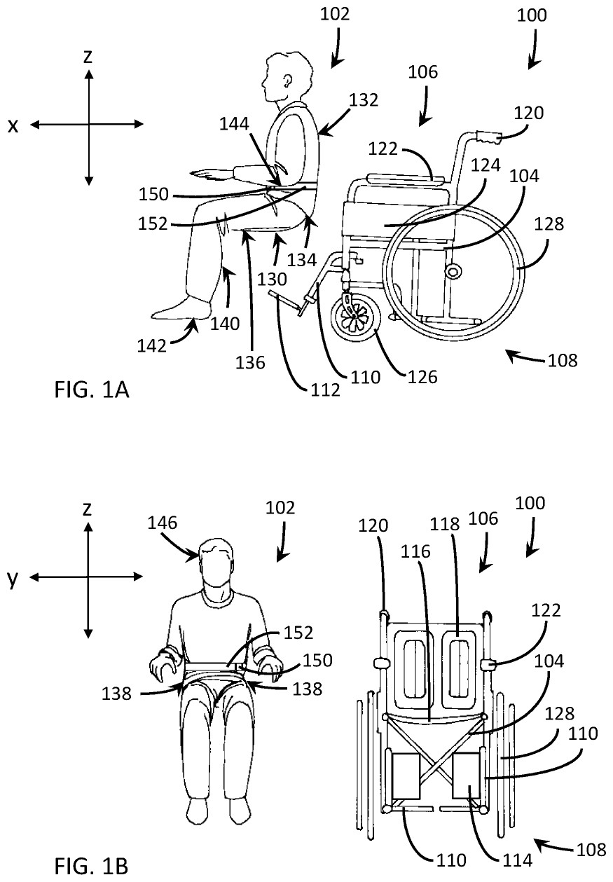

FIG. 1A is a side view drawing of an exemplary manual wheelchair 100 and a user 102. FIG. 1B is a front view drawing of the manual wheelchair 100 and the user 102. In these figures, the user 102 is shown with a posture when seated in the wheelchair 100, even though the user 102 is shown separated from the wheelchair, so that various features of the wheelchair 100 and portions of the user 102 can be more easily illustrated. Not every feature is shown in each view to reduce complexity.

Wheelchair 100 includes a frame assembly 104 that supports a seat 106. The frame is supported on a wheel system 108 for rolling locomotion along the ground. Foot and leg supporting brackets 110 are mounted to the front of left and right side frame portions. Foot supports 112 and leg supports 114 are attached to supporting brackets 110. The seat 106 includes a lower seat support 116 mounted to side frame portions for primary support of the user 102. A back support 118 is connected between upward extending members of left and right side frame extensions which terminate in attendant handles 120. A rubber grip may be used to facilitate manual grasping and moving of the handles 120. Arm supports 122 are mounted to the side frame portions. Side supports 124 may be mounted on the side portions of the frame below the arm supports 122.

The wheel system 108 includes right and left front wheels 126 which are pivotally mounted to the left and right side frame portions by casters. A pair of rear wheels 128 are mounted to the right and left side frame portions. The wheels typically include a hub which is rotatably mounted by bearings on an axle. A plurality of spokes (not shown) interconnect the hub with an outer rim. A tire, which may be a pneumatic, solid, or semi-solid tire, is mounted to the rim. Commonly, the tire has a tread or road engaging surface on its outermost periphery.

When the user 102 is supported in the wheelchair 100, various portions of the user 102 make contact with wheelchair surfaces, including several support surfaces, resulting in potential pressure points that could result in bedsores over time. In general terms, a bedsore is an ulceration of tissue deprived of adequate blood supply by prolonged pressure without adequate pressure relief. In particular, for example, with continued reference to FIGS. 1A and 1B, pressure points could include: where the user's thighs and buttocks 130 contact the seat support 116; where the user's thighs and buttocks 130 contact the seat support 116; where the user's shoulder blades 132 contact the back support 118; where the user's tailbone 134 contacts the seat support 116; where the user's backs of the knees 136 contact the seat support 116; where the user's hips 138 contact the side supports 124; where the user's calves 140 contact the leg supports 114; where the user's foot or heel 142 contact the foot supports 112; where the user's arm/elbow 144 contact the arm supports 122; where the user's head 146 contacts a head support (not shown); etc.

User movement can alleviate the pressure at a pressure point and allow for at least partial tissue reperfusion by momentary improved blood flow. In situations where the user initiates the movement, muscle contraction can offer further benefits. Techniques that move or monitor the support surfaces of the wheelchair are not able to ascertain user movement and are not always a suitable replacement for user movement. For example, a user 102 can move all or portions of the user's body while in the wheelchair 100 without any movement of the wheelchair support surfaces. If movement is only tracked by movement of the support surfaces, user movement independent of support surface movement will not be recognized and can lead to inaccurate pressure relief forecasting and management (including feedback). In addition, while movement of the wheelchair support surfaces may cause some movement of the user's body, the pressure at various pressure points may not be relieved.

A movement sensor 150 attached to the user 102 while the user 102 is supported by the wheelchair 100 can be used to detect the inertial movement of the user 102 and generate movement data. In some embodiments, the movement sensor 150 may include a gyroscope (gyro) and/or an accelerometer. One or more accelerometers may be used to detect movement in multiple planes/directions. Other types of sensors for detecting movement and generating movement data may also be used, including position sensors. In the embodiment shown in FIGS. 1A and 1B, the movement sensor is a wearable device that is attached to the user's torso with a belt 152. In other embodiments, as discussed in more detail below, multiple movement sensors 150 may be attached to various portions of the user's body to better detect various movements of the user 102 and/or the effects on one or more pressure points.

The movement data generated by the movement sensor 150 may be any suitable parameter and in any suitable format for tracking at least one aspect of user movement. For example, parameters may be a discrete motion sense, a duration, a distance, a rate, an acceleration, a position, or combinations thereof. Other metrics may also be tracked, including, for example, position, inclination, a change in position or inclination, etc. In particular embodiments, movement data from the movement sensor 150 may include one or more data: simply indicate that there was user movement; the length of movement time; the distance moved; the speed of the movement; the direction of movement; starting and finishing positions and inclinations; movement paths; movement gradients; etc. In some embodiments, the movement data may be relative to a reference point/object. For example, position and/or movement may be relative to a reference point on the wheelchair 100.

In one embodiment, movement sensor 150 detects front-to-back movement of the user in the x direction, side-to-side movement of the user in the y direction, and up-and-down movement of the user in the z direction. However, any combination of sensors and coordinate systems may be used to detect movement in any number of directions or dimensions. For example, in another embodiment, a movement sensor may be used to only track side-to-side and front-to-back user movement. In another embodiment, a movement sensor may be used to only track rotation about either the y or the x axis. Yet another embodiment might use a combination of both translation and rotation.

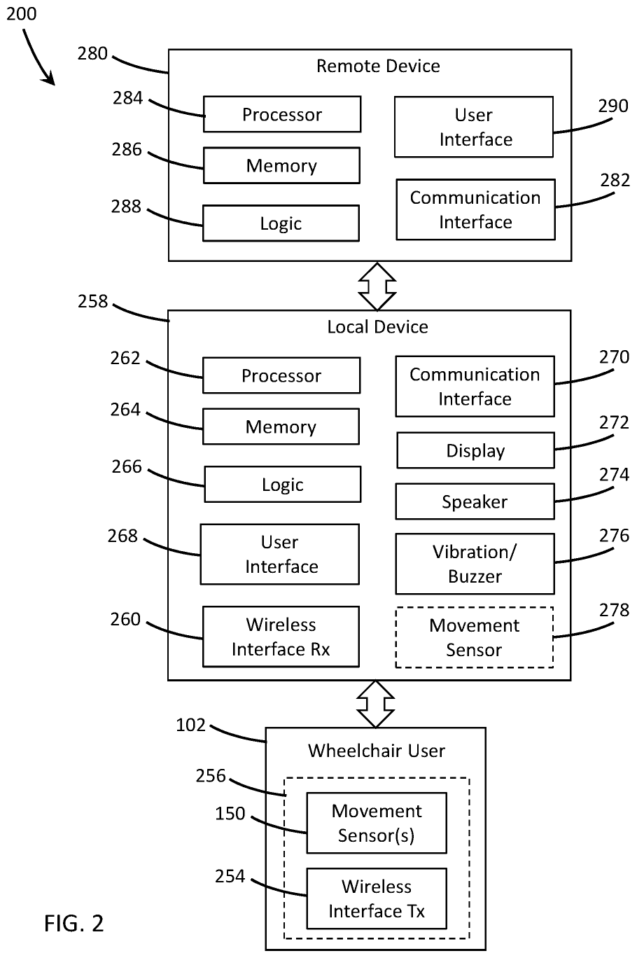

FIG. 2 is a block diagram of a pressure management system 200 associated with a user 102 of a wheelchair 100. The system 200 includes the movement sensor 150 attached to the user 102, as described above. This embodiment includes a wireless interface device 254, which is in operative communication with the movement sensor 150, to transmit the movement data to another device, preferably wirelessly. In some embodiments, a processor and/or memory (to cache data) may also be attached to the user with the capabilities of the local device mentioned below. In one embodiment, a wireless interface device may include sensing, processing, communication, storage capabilities, and/or other capabilities in one device. Various protocols may be used, including, for example, Bluetooth, IrDA, 802.11, 802.15, WLAN, WPA, WEP, Wi-Fi, and wireless broadband standards. In one embodiment, the wireless interface device 254 may be connected to the movement sensor 150 and attached to the user 102 as part of an integrated wearable device 256. In some embodiments, an output device (e.g., vibration device) may be included in the integrated wearable device 256 for feedback to the user 102. Any number of movement sensors may be connected to the wireless interface device 254.

As shown in FIG. 2, a local device 258 includes a wireless interface device 260 to receive the movement data transmitted from the wireless interface device 254. Although the movement data is transmitted from transmitter device 254 to receiver device 260, it should be understood that these wireless interface devices 254, 260 can both transmit and receive other data back and forth as necessary to establish communication and support other needs. Local device 258 can record, for example, pressure relief event type, time, date, and duration based on the movement data.

Local device 258 may also include a processor 262, a memory 264, logic 266, a user interface 268 (including inputs), communication interface 270, display 272, speaker 274, vibration/buzzer 276, and/or any other feature that may be used to develop and/or communicate notifications regarding pressure management. In various embodiments, the local device 258 may be a smart phone, tablet, laptop, or other computing device capable of receiving and processing the movement data and providing pressure management notifications. In other embodiments, the local device 258 may be a controller and/or programmer associated with the wheelchair.

Processor 262 processes the received movement data to generate pressure relief data, based on the inertial movement of the user 102, as described in detail below. Processor 262 may include a device or combination of devices that function as a processor, as defined above. Logic 266 may include software for processing movement data. For example, logic for the processing movement data can include generating pressure relief data based on the movement data, where the pressure relief data includes an indication of the pressure relief associated with at least one pressure point of the user. The memory 264 may store the logic 266, various constants/algorithms associated with the logic 266, predetermined times/durations, set-points, thresholds, protocols, notifications, etc. The memory 264 may be of any type or configuration, including, for example, local, remote, permanent, removable, centralized, shared, etc. The memory 264 may also store a database of pressure management routines, therapeutic schedules, plans, regimens, regimes, etc.

The user interface 268 may include various input devices, such as, for example, buttons, dials, mouse, keyboard, touch-pad, etc. The display 272 may include one or more displays, including, for example, monitors, readouts, LCDs, LEDs, etc. The communication interface 270 may include various devices suitable for any type of communication, including, for example, network connections (e.g., modem, LAN, WAN), wired (e.g., USB, Ethernet), wireless interfaces (as mentioned above), portable storage medium interfaces (e.g., flash drive ports (e.g., memory sticks, USB, multimedia, SD, compact flash)), etc., including for communication with remote devices and/or stations.

Display 272, speaker 274, and/or vibration/buzzer 276 are exemplary notification devices that may be used by the local device for communicating pressure relief information/notifications based on the generated pressure relief data via alerts.

The local device 258 may also include a movement sensor 278 with the same capabilities as mentioned above for movement sensor 150. In some embodiments, processing the movement data from the movement sensor 150 to determine user movement may include determining and accounting for the movement of the wheelchair 100. In an embodiment where the local device 258 resides on or is attached to the wheelchair 100, movement sensor 278 may be used for this purpose.

In other embodiments, the local device 258 may also communicate with a remote device 280 via communication interface 282. Remote device 280 may also include a processor 284, memory 286, logic 288, and user interface 290, with structures and capabilities similar to those mentioned above for local device 258. Remote device 280 may be a remote server. Local device 258 can post movement data and/or pressure relief data to the remote device 258 for storage, further access, processing, analysis, etc. In one embodiment, remote device 280 is used by the user 102 and/or a therapist to analyze the number and type of pressure reliefs performed and assessed relative to a prescribed pressure relief regime. In other embodiments, prescribed pressure relief regimes, updated algorithms/thresholds, new/revised logic, etc. can be communicated from the remote device 280 to the local device 258 for implementation for the user 102.

In various embodiments, the various components of system 200 may be separate components in operative communication with each other or may be integrated to various degrees. The degree of integration may range from discrete components sharing a common housing to full integration into one or more integrated components or devices with combined capabilities.

Returning now to the logic 266 for processing the movement data and generating pressure relief data that can indicate the pressure relief associated with a pressure point of the user 102. In some embodiments, various relationships between movements and pressure points may be established. In one embodiment, where only one movement sensor 150 is utilized, movement indicated by movement sensor 150 may be associated with pressure relief at one or more pressure points (PP). The pressure points may be, for example, contact points 130, 132, 134, 136, 138, 140, 142, 144, 146 described above.

In some embodiments, the movement data itself may be used to categorize a movement as a certain pressure relief event and/or type. For example the user starting to pitch forward to relieve pressure from the buttocks could be seen as both an angular acceleration about the y axis as well as linear acceleration in x and z axis. The release back into the chair would be seen as the opposite. In these embodiments, the pressure relief data may include the pressure relief event type, time, date, and/or duration.

In other embodiments, more complex processing may be utilized, including where multiple movements are combined, movements affect different pressure points differently, multiple sensors are combined, etc. For example, each movement of a user may be treated as a separate pressure relief event or movements over a period of time may be combined. Combining the movements and timings of movements may be additive, scaled, weighted, or follow any type of algorithm.

In another example, movement detected in the x direction by movement sensor 150 (M₁ₓ) may be used to generate pressure relief data at one or more pressure points (e.g., PP₁=a*M₁ₓ, PP₂=b*M₁ₓ, PP₃=c*M₁ₓ, etc., where a, b, and c may be constants defining the relationship between the sensor movement in the x direction and the expected amount of pressure relief at that specific pressure point). The movement data (M₁ₓ) may include any of the movement parameters mentioned above, for example, discrete sense, duration, distance, direction, etc. Similarly, movement detected in the y direction by movement sensor 150 (M_{1y}) may be used to generate pressure relief data at one or more of the same and/or different pressure points (e.g., PP₁=d*M_{1y}, PP₃=e*M_{1y}, PP₄=f*M_{1y}, etc., where d, e, and f may be constants defining the relationship between the sensor movement in the y direction and the expected amount of pressure relief at a different set of specific pressure points).

In another example, where more than one movement sensor 150 is utilized, movement indicated by the movement sensors 150 may also be associated with one or more pressure points (PP). For example, movements detected in the x direction by movement sensors 150 (M₁ₓ, M₂ₓ, etc.) may be used to generate pressure relief data at one or more pressure points (e.g., PP₁=a*M₁ₓ+b*M₂ₓ, PP₂=c*M₁ₓ+d*M₂ₓ, etc., where a, b, c, and d may be constants defining the relationship between the M₁ and M₂ sensor movements in the x direction and the expected amount of pressure relief at that specific pressure point). Similar formulas can be used for sensor movement in the y direction. As can be appreciated, the above formulas are only exemplary and any type of multi-dimensional mapping and weighting system may be used to establish these relationships. For example, in other embodiments, non-linear relationships between sensor movement and pressure relief may also be established.

Where algorithms are used, the expected pressure relief at any pressure point (PP) based on sensed movement may be determined using a variety of factors, including, for example, the distance between the sensor 150 and the PP, the relationship between the direction of sensor movement and the general plane of the PP, the anatomical relationship between the portion of the user's body where the sensor is attached and the PP (e.g., various physiological factors, such as, tissue, joints, etc., that translate one movement into another related movement in a connected body), etc. In other embodiments, various other factors may be considered by the algorithm, including, for example, lengths of limbs/torso, body mass index (BMI), other physiological factors, etc.

Regardless of the complexity of processing the movement data to generate the pressure relief data, the local device 258 can generate pressure relief information based on the pressure relief data. In one embodiment, the pressure relief information can be communicated from the local device 258 via a notification device associated with the local device (e.g., display 272, speaker, 274, and/or vibration/buzzer 276). In another embodiment, the local device 258 can send a signal containing the pressure relief information from the local device 258 to another device associated with the user 102, a local recipient, and/or a remote recipient. For example, the local device 258 can send a signal indicating the information to an output device (e.g., vibration device) included in the integrated wearable device 256 attached to the user 102 via the wireless interfaces 260, 254. Generally, the information can be communicated to the user 102, caregiver, therapist, management/monitoring system, and/or any other person and/or system associated with the user 102.

Regarding content, the pressure relief information can include any type of feedback associated with the pressure relief data. For example, the pressure relief information can include an instruction to the user 102 or a caregiver requesting user movement. In some embodiments, the instruction for user movement may include specific movements, durations, distances/extensions, etc. The instructions may be dictated by a particular pressure relief regime, as described above. In another example, the pressure relief information can include an indication of sufficient user movement, for example, in accordance with a particular pressure relief regime.

In one embodiment of system 200, a movement sensor 150 (e.g., accelerometer and/or gyro) is attached to the user 102 of a manual wheelchair 100, for example, on the user's belt or torso. The movement sensor 150 detects the movements of the user 102 from side-to-side and front-to-back. These movements, once detected, are transmitted wirelessly to the user's smart phone (local device 258) via the phone's Bluetooth connection as movement data. An app on the smart phone would send alerts to the user 102 when insufficient relief activities had been detected or a prescribed goal had been reached. As mentioned above, pressure relief data, such as event type, time, date and duration can be recorded by the app and posted to a remote server (remote device 280) for further analysis by the user 102 and/or therapist in view of a prescribed pressure relief regime.

In this manner, the app is able to initiate a series of movements for the user 102 while in the wheelchair 100. Instructions for movements of the user 102 are automatically generated by the system 200, typically separated by a predetermined or threshold period of time. In some embodiments, the maximum period of time between movements can vary. Movements by the user 102 can reset the timer tracking the time since the last movement. Thus, if the user 102 is adequately moving without reminders or instructions to do so, the system 200 may not have to notify the user 102 with any alerts to move. In some embodiments, the movement(s) by the user 102 may not be sufficient, as determined by the pressure relief data, thus resulting in alerts for additional movement. The maximum time intervals between movements can be programmed to correspond to a prescribed routine or regime. In this manner, the system 200 can verify sufficient user movement for various health benefits, such as improved circulation, a reduced likelihood of bedsores, etc.

Alternatively, in other embodiments, pressure relief movements can be aggregated to form a movement summary over predefined periods of time, for example, ranging from minutes to days. This aggregated summary can then also be used to generate alerts for goal achievement or risk of non-compliance to prescribed pressure relief regimes.

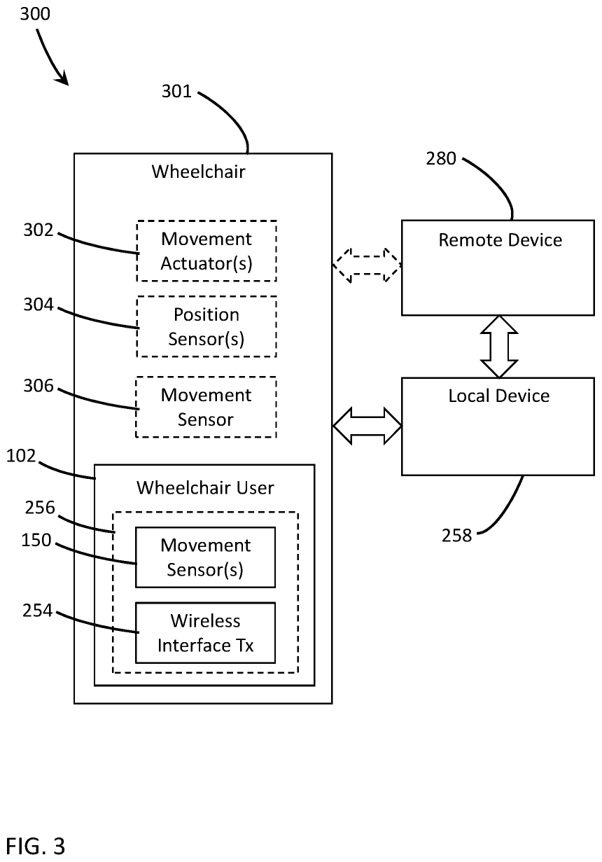

FIG. 3 is a block diagram of a pressure management system 300 associated with a user 102 of a powered wheelchair 301. The system 300 includes the movement sensor 150 attached to the user 102, as described above. This embodiment also includes local device 258 (and its components) and remote device 280 (and its components), as described above. However, in this embodiment, certain features of powered wheelchair 301 may be utilized by system 300. In addition to the embodiment where the local device 258 is a smart phone, in other embodiments of system 300, the local device 258 may include a controller or programmer of the powered wheelchair 301. Communication devices (not shown) of the powered wheelchair 301 may also be utilized by the system 300 to communicate with the local device 258 and/or the remote device 280.

In particular, for example, the powered wheelchair may include movement actuator(s) 302, position sensor(s) 304, and a movement sensor 306. Movement actuator(s) 302 may be associated with one or more user support surfaces, including, for example, a seat bottom, a seat back, foot/leg supports, etc. (which may be similar to one or more of the various user supports shown above in FIG. 1) for powered and/or automated movement of the support surface. Position sensor(s) 304 may also be associated with one or more of the user support surfaces and/or movement actuators 302 for determining/tracking the position of the support surface. Movement sensor 306 may be used to track the movement of the wheelchair 301. To the extent that the logic of system 300 utilizes the movement of the wheelchair 301 in combination with user movement sensor 150 to determine the independent movement of the user 102, movement sensor 306 of the powered wheelchair (which may include, for example, one or more gyros and/or accelerometers) may be utilized.

In the embodiments of system 300, when the pressure relief data indicates that the user 102 has insufficient movement and needs to move, movement actuators 302 may be utilized to move the user 102, including, in some embodiments, automatically. For example, if the pressure relief information includes an instruction for user movement, the user 102 or caregiver can move the user by commanding the movement actuators 302 to move to a particular position, either manually or with predefined positions. In another example, the instruction for user movement can communicate with an auto-positioning system to move the user 102 automatically. However, it should be noted that in these embodiments, the user movement sensor 150 is utilized to monitor actual user movement.

In some embodiments, the system 300 can implement a method of automatically positioning user support surfaces of a powered wheelchair through a series and/or sequence with user movement feedback. For example, U.S. patent application Ser. No. 14/802,221, now U.S. Pat. No. 9,522,091, and titled “Method and apparatus for automated positioning of user support surfaces in power driven wheelchair,” is hereby incorporated in its entirety.

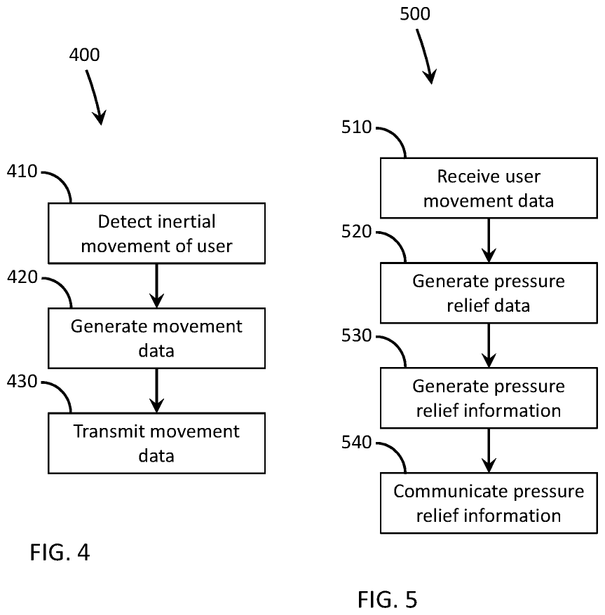

FIGS. 4-7 are block diagrams of exemplary methodologies associated with the apparatus and systems above. The exemplary methodologies may be carried out in logic, software, hardware, or combinations thereof. In addition, although the methods are presented in an order, the blocks may be performed in different orders. Further, additional steps or fewer steps may be used.

FIG. 4 is a block diagram of an exemplary method 400 for detecting and transmitting inertial movement of a user of a wheelchair. Method 400 can be executed using the apparatus and systems mentioned above. First, at step 410, the method detects the inertial movement of the user in the wheelchair using a movement sensor configured to be attached to the user while the user is supported by the wheelchair. Then, at step 420, the method generates movement data indicative of the inertial movement of the user in the wheelchair. At step 430, the movement data is transmitted to a local device.

FIG. 5 is a block diagram of an exemplary method 500 for generating pressure management information associated with a user of a wheelchair. Method 500 can be executed using the apparatus and systems mentioned above. First, at step 510, the method receives movement data indicative of the inertial movement of the user in the wheelchair. Then, at step 520, the method generates pressure relief data based on the movement data. In some embodiments, step 520 can include determining a pressure relief associated with at least one pressure point of the user. As discussed above, determining the pressure relief associated with a pressure point of the user can include combining movement data associated with a plurality of movements detected by one movement sensor and/or combining movement data associated with a plurality of movements detected by a plurality of movement sensors.

At step 530, the method generates pressure relief information based on the pressure relief data. Next, at step 540, the pressure relief information is communicated using a notification device. In some embodiments, step 540 can include communicating an instruction for user movement or an indication of sufficient user movement. In some embodiments, the method can also include communicating at least one of the movement data and the pressure relief data to a remote device.

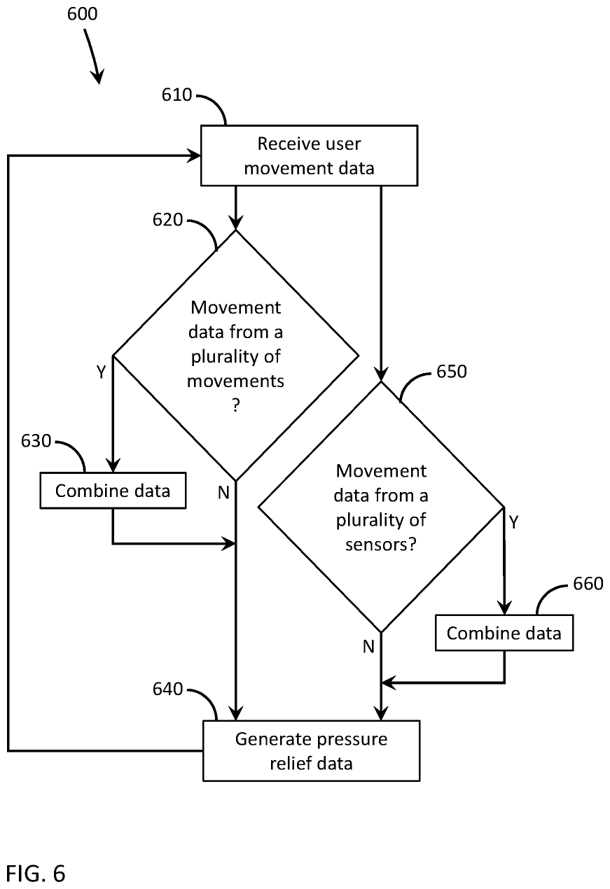

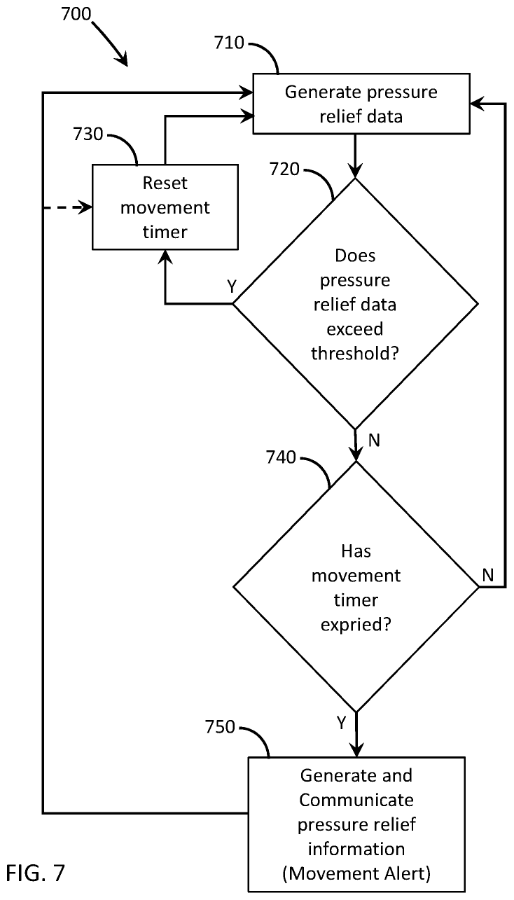

FIG. 6 is a block diagram of an exemplary method 600 for generating pressure relief data. First, at step 610, the method receives movement data indicative of the inertial movement of a user in a wheelchair. Then, at step 620, the method determines if the total and/or incremental movement data is from a plurality of movements. In one embodiment, as part of the pressure management method for determining if movement is adequate for pressure relief, a running total of movements may be logged and combined to determine an accumulated pressure relief in general or for particular pressure points. For example, a timer for a movement alert may not reset until a certain minimum amount of movement is detected, as described below with reference to FIG. 7. If there is movement data representing a plurality of movements, the method proceeds to step 630, where the movement data is combined and pressure relief data is generated or updated at step 640. If the movement data is not from a plurality of movements, the method proceeds directly to step 640 to generate the pressure relief data.

At step 650, the method determines if the total and/or incremental movement data is from a plurality of sensors. In one embodiment, as part of the pressure management method for determining if movement is adequate for pressure relief, a running total of movements may be logged and combined to determine an accumulated pressure relief in general or for particular pressure points. If there is movement data from a plurality of sensors, the method proceeds to step 660, where the movement data is combined and pressure relief data is generated or updated at step 640. If the movement data is not from a plurality of sensors, the method proceeds directly to step 640 to generate the pressure relief data.

FIG. 7 is a block diagram of an exemplary method 700 for generating and communicating pressure relief information. First, at step 710, the method generates pressure relief data indicative of the pressure relief associated with movements of a user in a wheelchair. If the pressure relief meets or exceeds the threshold level, the method proceeds to step 730, where a movement timer is reset. In some embodiments, achieving the required level of movement may also generate pressure relief information in the form of an achievement alert notifying the user, caregiver, and/or therapist that sufficient movement was reached by the user. If the pressure relief does not meet or exceed the threshold level, the method proceeds to step 740 to determine if the movement timer has reached a threshold. If the movement timer has not expired, the method loops back and continues to generate and/or update the pressure relief data at 710. If the movement timer has expired, the method proceeds to step 750 where pressure relief information in the form of a movement alert is generated and communicated to notify the user, caregiver, and/or therapist instructing user movement since insufficient movement was achieved by the user within the prescribed time frame. Following the movement alert at 750, the method loops back and continues to generate and/or update the pressure relief data at 710, generating additional periodic alerts while awaiting the requested movement. In an alternative embodiment, the method may proceed to step 730, where the movement timer is reset after one or more movement alerts are sent.

While the present invention has been illustrated by the description of embodiments thereof and while the embodiments have been described in considerable detail, it is not the intention of the applicant to restrict or in any way limit the scope of the appended claims to such detail. Additional advantages and modifications will readily appear to those skilled in the art. Therefore, the invention, in its broader aspects, is not limited to the specific details, the representative apparatus and methods, and illustrative examples shown and described. Accordingly, departures may be made from such details without departing from the spirit or scope of the applicant's general inventive concept. While the embodiments discussed herein have been related to the systems and methods discussed above, these embodiments are intended to be exemplary and are not intended to limit the applicability of these embodiments to only those discussions set forth herein. The control systems and methodologies discussed herein may be equally applicable to, and can be utilized in, other systems and methods.

We claim:

1. An apparatus for detecting and transmitting inertial movement of a user of a wheelchair, the apparatus comprising:
at least one movement sensor configured to be attached to the user while the user is supported by the wheelchair, wherein the movement sensor detects inertial movement of the user and generates movement data; and
a wireless interface device in operative communication with the at least one movement sensor, wherein the wireless interface device transmits the movement data wirelessly to a receiver associated with the apparatus.

2. The apparatus of claim 1, wherein the at least one movement sensor comprises at least one of a gyro and an accelerometer.

3. The apparatus of claim 1, wherein the movement data comprises side-to-side and front-to-back movement of the user in the wheelchair.

4. The apparatus of claim 1, wherein the at least one movement sensor is attached to the torso of the user.

5. The apparatus of claim 1, wherein the at least one movement sensor comprises a plurality of movement sensors attached to the user at a plurality of locations.

6. The apparatus of claim 1, wherein wireless interface device transmits the movement data to the receiver using a Bluetooth protocol.

7. A pressure management system for a user of a wheelchair, the system comprising:
a user monitoring apparatus, comprising:
at least one movement sensor configured to be attached to the user while the user is supported by the wheelchair, wherein the movement sensor detects inertial movement of the user and generates movement data; and
a first wireless interface device in operative communication with the at least one movement sensor, wherein the first wireless interface device transmits the movement data wirelessly to another wireless interface associated with the apparatus;
a local device, comprising:
a second wireless interface device, wherein the second wireless interface device receives the movement data wirelessly from the first wireless interface;
a processor for processing the movement data, wherein the movement data is processed to generate pressure relief data based on the inertial movement of the user; and
a notification device for communicating pressure relief information based on the pressure relief data.

8. The system of claim 7, wherein the local device further comprises a memory comprising logic for generating the pressure relief data based on the inertial movement of the user, and wherein the pressure relief data comprises an indication of pressure relief associated with at least one pressure point of the user.

9. The system of claim 8, wherein the inertial movement of the user comprises a plurality of movements detected by one movement sensor.

10. The system of claim 8, wherein the inertial movement of the user comprises a plurality of movements detected by a plurality of movement sensors.

11. The system of claim 8, further comprising logic for generating pressure relief information based on the pressure relief data, and wherein the pressure relief information comprises an instruction for user movement.

12. The system of claim 8, further comprising logic for generating pressure relief information based on the pressure relief data, and wherein the pressure relief information comprises an indication of sufficient user movement.

13. The system of claim 7, further comprising a remote device in communication with the local device, wherein the local device communicates at least one of the movement data and the pressure relief data to the remote device.

14. A method of generating pressure management information associated with a user of a wheelchair, comprising:
receiving movement data indicative of the inertial movement of the user in the wheelchair using a wireless interface;
generating pressure relief data based on the movement data;
generating pressure relief information based on the pressure relief data; and
communicating the pressure relief information using a notification device.

15. The method of claim 14, further comprising:
detecting the inertial movement of the user in the wheelchair using at least one movement sensor configured to be attached to the user while the user is supported by the wheelchair;
generating the movement data indicative of the inertial movement of the user in the wheelchair; and
transmitting the movement data to a local device.

16. The method of claim 14, wherein generating pressure relief data comprises determining a pressure relief associated with at least one pressure point of the user.

17. The method of claim 16, wherein determining the pressure relief associated with at least one pressure point of the user comprises combining movement data associated with a plurality of movements detected by one movement sensor.

18. The method of claim 16, wherein determining the pressure relief associated with at least one pressure point of the user comprises combining movement data associated with a plurality of movements detected by a plurality of movement sensors.

19. The method of claim 14, wherein communicating the pressure relief information comprises communicating an instruction for user movement or an indication of sufficient user movement.

20. The method of claim 14, further comprising communicating at least one of the movement data and the pressure relief data to a remote device.
